(19) United States
(12) Patent Application Publication
(10) Pub. No.: US 2023/0054551 A1
(43) Pub. Date: Feb. 23, 2023

(54) ORAL PHARMACEUTICAL COMPOSITION AND METHOD FOR DELIVERING NITRIC OXIDE TO A PATIENT'S CIRCULATORY SYSTEM OR BRAIN

(71) Applicant: National Tsing Hua University, Hsinchu (TW)

(72) Inventors: Tsai-Te LU, Hsinchu (TW); Cheng-Ru WU, Hsinchu (TW)

(21) Appl. No.: 17/579,053

(22) Filed: Jan. 19, 2022

(30) Foreign Application Priority Data
Aug. 7, 2021 (TW) 110129206

(51) Int. Cl. A61K 31/295; A61K 9/00; A61P 25/28

(52) U.S. Cl. CPC A61K 31/295; A61K 9/0053; A61P 25/28

(57) ABSTRACT

A pharmaceutical composition for oral administration and a use of a pharmaceutical composition for preparing oral medication for delivering nitric oxide to the patient's circulatory system or brain are provided. The pharmaceutical composition mainly includes a dinitrosyl iron complex and a pharmaceutically acceptable excipient. The pharmaceutical composition delivers nitric oxide to the patient's circulatory system or brain to stimulate neurogenesis or regulate blood sugar.

### CROSS REFERENCE TO RELATED APPLICATION

This application claims the benefit of Taiwan Patent Application Serial No. 110129206 filed on Aug. 7, 2021, the subject matter of which is incorporated herein by reference.

### BACKGROUND OF THE INVENTION

### 1. Field of the Invention

The present invention relates to an oral pharmaceutical composition and a method for delivering nitric oxide to a patient's circulatory system; particularly, to an oral pharmaceutical composition for delivering nitric oxide to a patient's circulatory system or brain and a method for delivering nitric oxide to a patient's circulatory system for stimulating neurogenesis or regulating blood sugar.

### 2. Description of Related Art

Nitric oxide (NO) is a biologically active gas molecule which has been proved to have many physiologically functions, such as anti-inflammatory and anti-apoptosis. At present, the FDA approved nitric oxide reagents can be administered via sublingual administration, inhalation, intravenous injection or intravenous infusion. Oral administration is relatively a convenient route for administration; however, reagent for oral administration has not been developed because nitric oxide is unstable.

Moreover, the recovery of the circulatory system and the brain from neurodegenerative diseases and ischemic stroke is critical because the clinical treatments cannot stimulate nerve repair or neurogenesis but only prevent the worsening of the condition. Given the condition of unstable blood sugar in diabetic patients, the most widely used treatment is giving them insulin; however, insulin needs to be administered intravenously.

Accordingly, the present invention provides an oral administered pharmaceutical composition including double nitroso iron complex, the oral pharmaceutical composition delivers nitric oxide to the body by oral administration for repairing the brain damages after neurodegenerative diseases or ischemic stroke or regulating blood sugar for diabetes.

### SUMMARY OF THE INVENTION

The present invention provides an oral pharmaceutical composition, comprising a double nitroso iron complex of formula (I) (DNIC-1), and a pharmaceutically acceptable excipient:

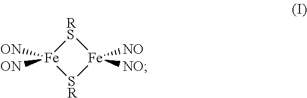

wherein R represents a C₁-C₅ hydroxyl group.

In one embodiment, the pharmaceutically acceptable excipient is at least one selected from a group consisting of mannitol, xylitol, sorbitol, maltol, maltitol, lactose, sucrose, maltose, and mixture thereof.

In one embodiment, a dosage form of the oral pharmaceutical composition is a tablet, a capsule, or an oral solution.

In one embodiment, R represents a C₂ hydroxyl group.

The present invention also provides a method for delivering nitric oxide to a patient's circulatory system or brain, comprising: orally administering the oral pharmaceutical composition to the patient.

In one embodiment, delivering nitric oxide to a patient's circulatory system or brain for stimulating neurogenesis or regulating blood sugar.

In one embodiment, delivering nitric oxide to a patient's circulatory system or brain for stimulating neurogenesis refers to a treatment of a neurodegenerative disease or an ischemic stroke.

In one embodiment, the neurodegenerative disease is at least one selected from a group consisting of amyotrophic lateral sclerosis (ALS), Parkinson's disease (PD), Alzheimer's disease (AD), Huntington's disease (HD), frontotemporal dementia (FTD), spinocerebellar ataxias (SCA), Machado-Joseph disease (MJD), Dentatorubropallidoluysian atrophy (DRPLA), spinal and bulbar muscular atrophy (SBMA), and fragile X-associated tremor and ataxia syndrome (FXTAS).

In one embodiment, regulating blood sugar refers to a treatment of diabetes.

### BRIEF DESCRIPTION OF THE DRAWINGS

FIG. 1 is an EPR spectrum of mice's stomach of one embodiment of the present invention;

FIG. 2 is a time-dependent biodistribution study of DNIC-1 in the mice's stomach of one embodiment of the present invention;

FIG. 3 is an EPR spectrum of mice's plasma of one embodiment of the present invention;

FIG. 4 shows the pharmacokinetic study of DNIC-1 in the plasma of one embodiment of the present invention;

FIG. 5 shows the time-dependent biodistribution study of nitric oxide in the mice's brain of one embodiment of the present invention;

FIG. 6 shows the cell viability assay of the BV-2 of one embodiment of the present invention;

FIG. 7 shows the cell viability assay of the N2A of one embodiment of the present invention;

FIG. 8 shows the secretion of TNF-σc in LPS-activated BV-2 of one embodiment of the present invention;

FIG. 9 shows the concentration of nitric oxide in the supernatant culture media for BV-2 cells of one embodiment of the present invention;

FIG. 10 shows the body weight observed in aging mice of one embodiment of the present invention;

FIG. 11 shows serum glucose observed in aging mice of one embodiment of the present invention;

FIG. 12 shows the serum triglyceride (TG) observed in aging mice of one embodiment of the present invention;

FIG. 13 shows the s serum total cholesterol observed in aging mice of one embodiment of the present invention;

FIG. 14 shows the test result of the aging mice in Morris water-maze task of one embodiment of the present invention;

FIG. 15 shows the Western blot analysis of aging mice of one embodiment of the present invention;

FIG. 16 shows the GOT level in the plasma of aging mice of one embodiment of the present invention;

FIG. 17 shows the GPT level in the plasma of aging mice of one embodiment of the present invention;

FIG. 18 shows the creatinine level in the plasma of aging mice of one embodiment of the present invention; and

FIG. 19 shows the urea level in the plasma of aging mice of one embodiment of the present invention;

### DETAILED DESCRIPTION OF THE PREFERRED EMBODIMENT

Hereafter, examples will be provided to illustrate the embodiments of the present invention. The advantages and effects of the invention will become more apparent from the disclosure of the present invention. Other various aspects also may be practiced or applied in the invention, and various modifications and variations can be made without departing from the spirit of the invention based on various concepts and applications.

[Animals]

C57BL/6JNarl (7 weeks old, male) for pharmacokinetic and biodistribution experiments of the present embodiment were purchased from National Laboratory Animal Center (Taipei, Taiwan) and BioLASCO Taiwan Co., Ltd. (Taipei, Taiwan), whereas C57BL/6JNarl (12-15 months old, male) for aging mice with metabolic syndrome were obtained from National Laboratory Animal Center (Taipei, Taiwan). The animals were housed under a 12-h light/12-h dark cycle at a controlled temperature (22±2° C.) and humidity (40±10%) and provided with food and water according to the Institutional Animal Care and Use Committee.

[Evaluation of Oral Delivery of Nitric Oxide to the Blood and Brain by DNIC-1]

After the C57BL/6JNarl male mice were fed with DNIC-1 (13.25 mg/kg) by oral gavage for 0.1, 0.5, 1, 2, 4, and 8 h, 200 μL of blood was collected from the facial vein and mixed with 50 μL of heparin (1,000 U/mL, Sigma-Aldrich). The isolated blood was centrifuged at 3000 rpm for 10 min under 4° C. before the Electron paramagnetic resonance spectrum (EPR) for 100 μL of the supernatant solution was measured. Based on the calibration curve of DNIC-1 in the blood described above, the pharmacokinetic curve of DNIC-1 was established from three independent experiments. Time-dependent decay of EPR signal intensity after oral administration of DNIC-1 for 0.5 h was then fit to pseudo-first-order kinetics to determine the in vivo half-life for DNIC-1 in the blood.

Time-dependent biodistribution study of DNIC-1 was evaluated similarly. After the C57BL/6JNarl male mice were fed with DNIC-1 (13.25 mg/kg) by oral gavage for 0.1, 0.5, 1, 2, 4, 8, 16, and 24 h, the mice were then sacrificed. Stomach, small intestine, large intestine, liver, kidney, and brain were collected respectively and homogenized in lysis buffer (10 mM Tris-HCl, 1% Triton X-100, 0.1% SDS, 0.1% sodium deoxycholate, and 140 mM NaCl). The tissue lysates were kept on ice for 30 min and centrifuged at 25,000 g at 4° C. for 30 min before the EPR spectrum for 100 μL of the supernatant solution was measured. Concentrations of DNIC-1 in each organ was further determined based on the calibration curve derived from the addition of different concentration of DNIC-1 into the corresponding organs.

Using the brain lysates obtained as described above, the concentration of nitric oxide was further determined by Nitrate/Nitrite Colorimetric Assay Kit to evaluate the time-dependent biodistribution study of nitric oxide in the brain after treatment of DNIC-1. The testing procedure is described as below. 500 μL aliquot of brain lysate was added into spin column (molecular weight cut off [MWCO] 3 kDa, Merck) and centrifuged at 29000 rpm for 10 min to remove the proteins from the brain lysates. Concentration of nitric oxide in this protein-free brain lysate was further determined using Nitrate/Nitrite Colorimetric Assay Kit.

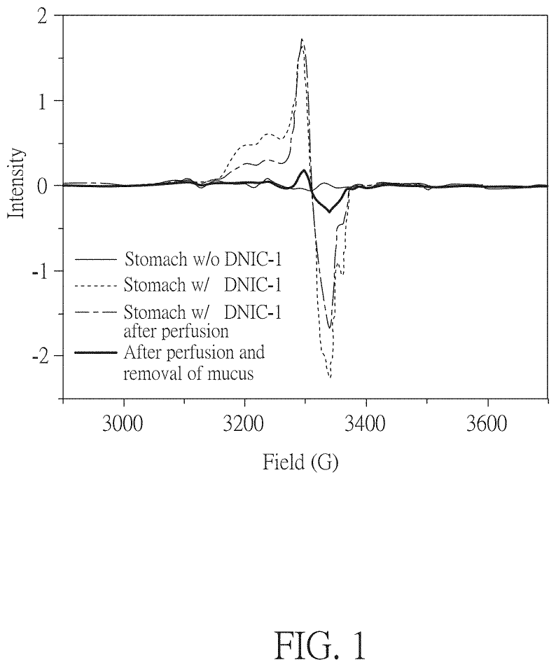

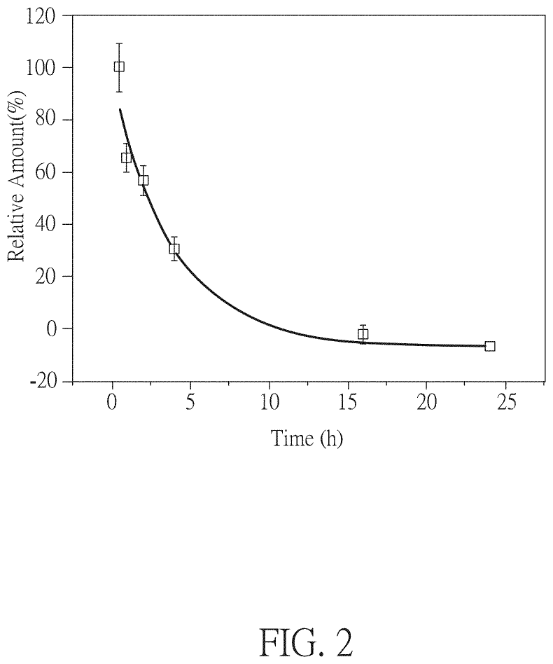

The test results were described as below. Refer to FIG. 1, after oral administration of DNIC-1 to mice, an EPR signal at g=2.041, 2.031, and 2.012 observed in the stomach demonstrates the instant formation of protein-bound DNIC containing a mononuclear S=½{Fe(NO)₂}⁹ unit, whereas this EPR feature remains active even after perfusion of the stomach. Significant decrease of this distinctive EPR feature was observed after further removal of mucus layer on the inner surface of stomach. Therefore, this distinctive EPR signal featured by gastrointestinal mucus discloses the rapid association of DNIC-1 to mucin and assembly of mucin-bound DNIC embedded in the gastrointestinal mucus. As shown in FIG. 2, based on the time-dependent biodistribution study of DNIC-1, a half-life of 2.7±0.6 h in the stomach was characterized, which shows significant contrast to that of 0.4±0.1 h for DNIC-1 in SGFsp. As a consequence, mucoadhesive nature of DNIC-1, derived from its rapid transformation into mucin-bound DNIC, provides a mechanism for protection and temporal storage encapsulated in the gastrointestinal mucus layer of the stomach.

In order to investigate the stability of DNIC-1 in the plasma isolated from mice, mice were anesthetized with intraperitoneal injection of 2.5% tribromoethanol (Avertin) in DEPC water and sacrificed. After a V-shaped cut through the skin and abdominal wall about 1 cm caudal to the last rib, 700-1000 μL of blood samples were taken from the heart of each mice using 25G needle before addition of 10% (v/v) heparin (1,000 U/mL, Sigma-Aldrich) to avoid the coagulation. To 4 mL of this fresh isolated blood sample, 4 μL of DMSO stock solution of DNIC-1 (25 mM) was added before this solution was incubated at 37° C. for 0, 1, 2, 4, 8, 24, and 28 h, respectively. At each time point, 400-μL aliquot of this solution was transferred to a 1.5-mL Eppendorf tube and centrifuged at 3000 rpm for 10 min before the EPR spectrum for 100 μL of the supernatant solution was measured. Based on the calibration curve derived from addition of 0, 1, 2, 5, 10, 20, and 50 μM of DNIC-1 in the blood, time-dependent degradation of protein-bound DNIC was then fit to pseudo-first-order kinetics to determine the half-life for DNIC-1 in the blood isolated from mice. Three independent experiments were executed to measure the average half-life for DNIC-1 in the blood isolated from mice.

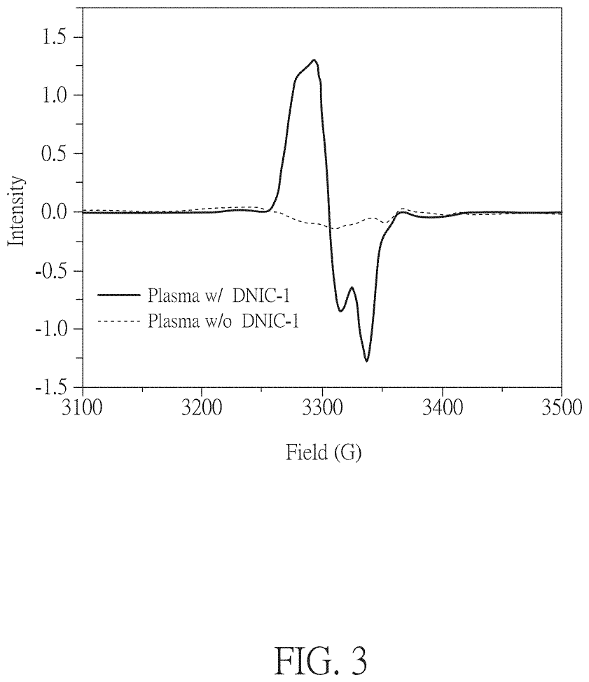

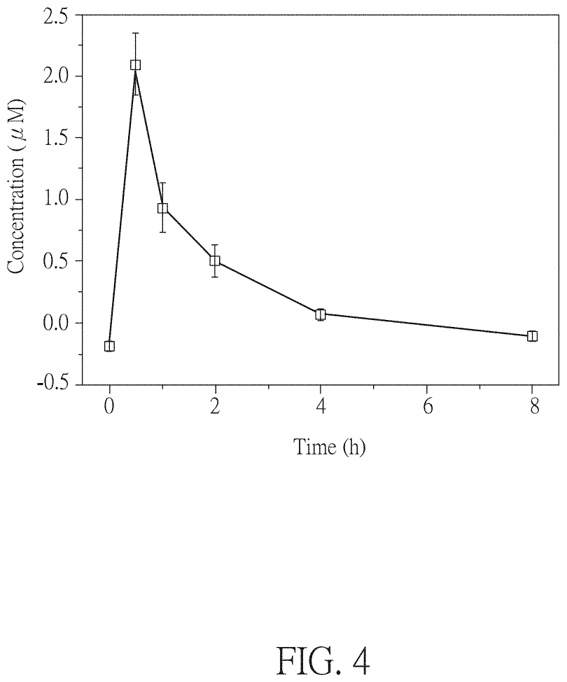

Next, as illustrated in FIG. 3, the albumin-bound DNIC-1 found in the plasma had proved that DNIC-1 was absorbed by the digestive system and entered into the blood system. As shown in FIG. 4, the pharmacokinetic study of DNIC-1 in the plasma reflects on a half-life of 0.9±0.2 h, which is comparable to the reported value of 1.2 h after intravenous injection of DNIC-1. Through the comparison with the area under curve derived from pharmacokinetics of DNIC-1 after intravenous injection, the pharmacokinetics of DNIC-1 after oral administration characterizes an oral bioavailability of 6.5%. As opposed to the half-life of 4.5±0.2 h for DNIC-1 in isolated plasma, the half-life of 0.9±0.2 h for DNIC-1 in the plasma after oral administration is ascribed to its uptake by organs such as the liver and the kidney.

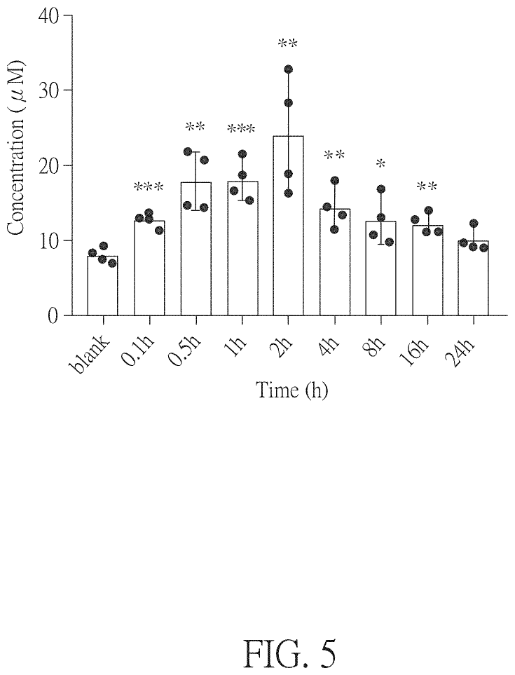

In contrast to the EPR observed in the brain isolated from the mice without the treatment of DNIC-1, the formation of the EPR signal at g˜2.03 with hyperfine structure supports the successful delivery of double nitroso iron unit (DNIU) [Fe(NO)₂] into the brain by oral administration of DNIC-1. Subsequent release of nitric oxide from the DNIU [Fe(NO)₂] results in the sustained elevation of NO level in the brain, whereas the concentration of NO recovers to its original level after oral administration of DNIC-1 for 24 h (FIG. 5).

[In Vitro Anti-Inflammation Effect and Pro-Neurogenic Effect of DNIC-1]

Based on the fact that DNIC-1 can be delivered and release nitric oxide in the brain into the brain, the in vitro anti-inflammation effect and pro-neurogenic effect of DNIC-1 in microglial cells (BV-2) and neuroblast cells (N2A) were further evaluated by the following method. Briefly, BV-2 microglial cells were seeded into a 24-well plate at a density of 1×10⁵ cells/well and incubated overnight. On the next day, BV-2 microglial cells were co-treated with LPS (1 μg/ml) and DNIC-1 at different concentrations (0, 0.5, 1, and 5 μM) and incubated for 6 h.

The concentration of TNF-α (pg/mL) was measured by mouse TNF-α ELISA kits (RayBiotech) following the manufacturer's protocol.

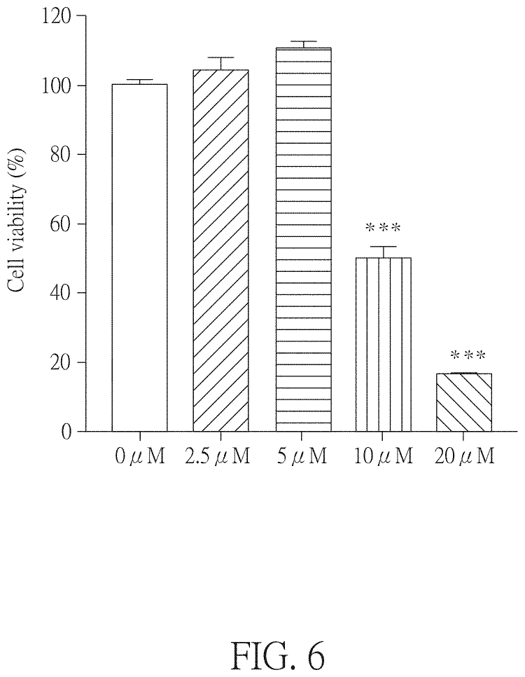

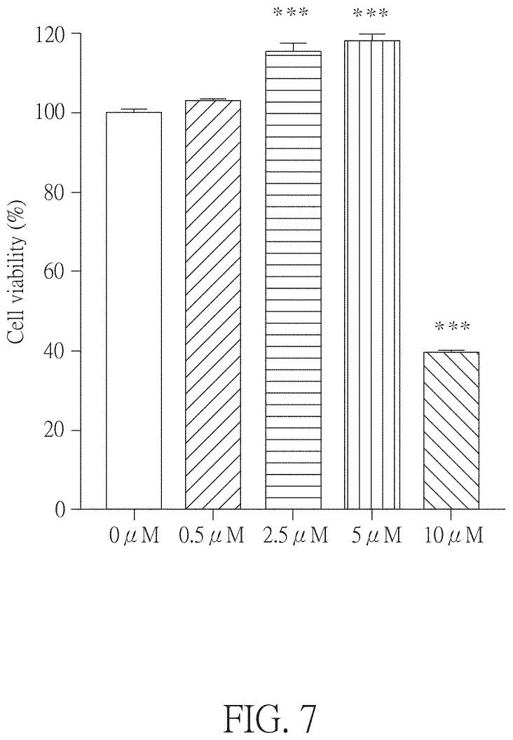

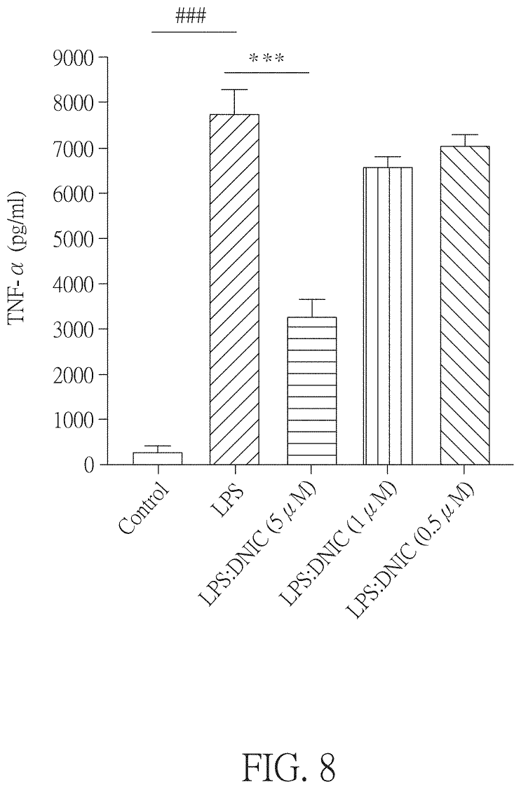

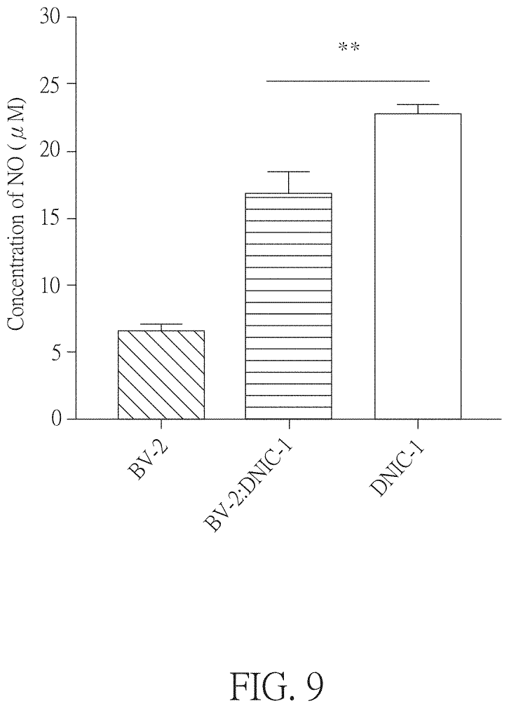

The results were shown in FIG. 6 and FIG. 7, DNIC-1 exhibits the IC₅₀ values of 11.7±3.6 μM and 9.8±0.6 μM toward BV-2 and N2A cells, respectively. As shown in FIG. 8, upon treatment of LPS (1 μg/m), activation of BV-2 cells into a pro-inflammation state was evidenced by the elevated generation and secretion of cytokine TNF-α. Refer to FIG. 8 and FIG. 9, the anti-neuroinflammation effect was evidenced according to the suppressed level of secreted TNF-α upon treatment of DNIC-1. On the other hand, as shown in FIG. 7, the proliferation of neuroblast cells triggered by 2.5 μM or 5 μM of DNIC-1 demonstrates its potential application to neurogenesis.

[Improvements of Neurodegenerative Diseases and Metabolic Syndrome by DNIC-1]

Inspired by the anti-inflammation effect and pro-neurogenic effect of DNIC-1, the potential for treating neurodegenerative disease and metabolic syndrome of DNIC-1 was further investigated.

C57BL/6JNarl (12-15 months old, male) were randomly divided into three groups, and each group received one of the following treatments for 16 weeks: (1) CD group, the control diet group was fed with LabDiet 5001 (containing 13.5% of kilocalories from fat and 3.7% of kilocalories from sucrose) freely; (2) WD group, the western diet group was fed with Diet-Induced Obesity Rodent Purified Diet w/45% kilocalories from fat and 17% kilocalories from sucrose freely; (3) WD:NO group, the WD:NO group was fed with the same Diet as WD group and additional treatment of DNIC-1 (2.65 mg/kg) via oral administration in a daily manner.

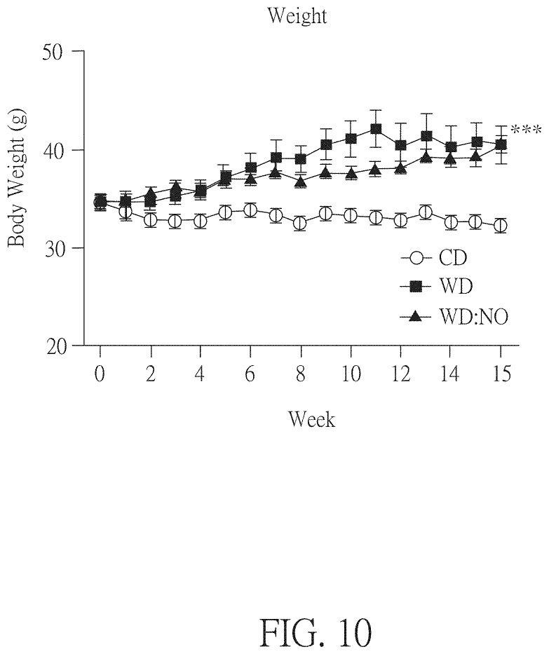

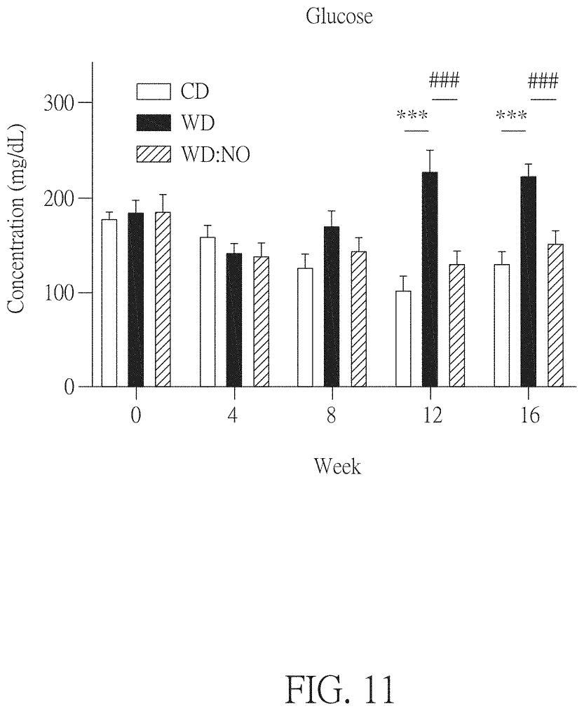

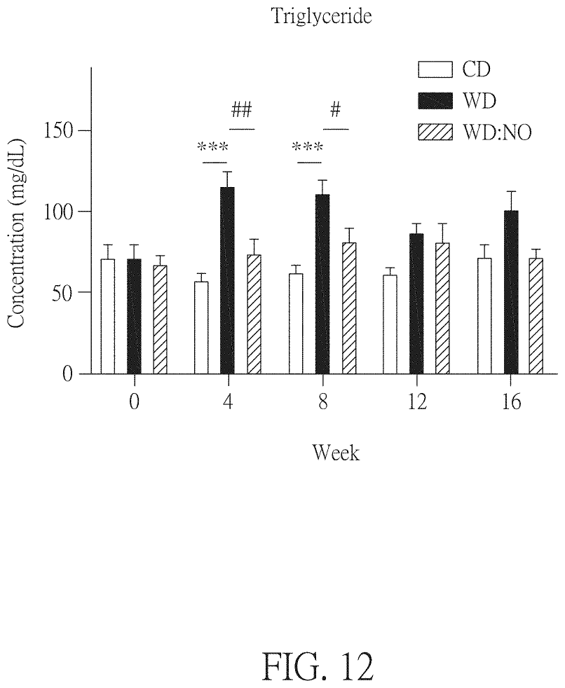

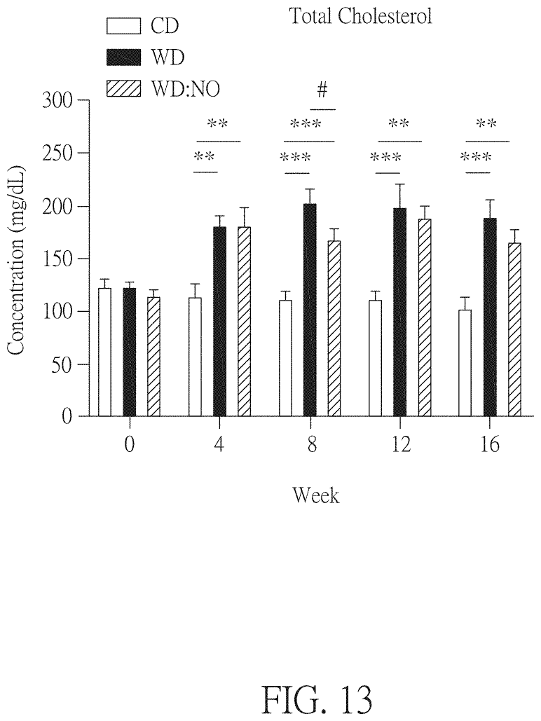

Utilization of aging mice under western diet (WD group) as a disease model for mild cognitive impairment, whilst daily treatment of DNIC-1 at 2.65 mg/kg via oral administration (WD:NO group) was performed to investigate the therapeutic efficacy of DNIC-1. Blood samples were collected every four weeks to measure the serum glucose, triacylglycerol (TG), and total cholesterol. After coagulation, the mouse sera were collected from blood samples by centrifugation at 10,000 g for 20 min at 4° C. The analysis results of these samples were shown in FIG. 10 to FIG. 13. As opposed to the aging mice under control diet (CD group), significant elevation of body weight and serum glucose/triglyceride/total cholesterol observed in the WD group indicates the successful induction of obesity and metabolic syndrome, which was reported to correlate with cognitive impairment. Through the comparison between WD and WD:NO groups, a decrease of serum glucose level induced by daily treatment of DNIC-1 is noticed.

After treatment for 16 weeks, cognitive functions including spatial learning and memory of the aging mice in CD, WD, and WD:NO groups were further assessed using Morris water maze task.

The Morris Water Maze test was performed with the apparatus consisted of a circular pool (diameter: 120 cm, height: 45 cm) filled with water (depth: 30 cm, 25±0.5° C.) and an escape platform (diameter: 10 cm) placed in the middle of one of the quadrants, 1 cm below the water surface, and equidistant from the sidewall and the middle of the pool. Each mouse was subjected to a series of four trials per day. For each trial, the mouse was placed into the water maze at four different positions. The trial began by placing the animal in the water facing the wall of the pool as one of the starting points. If the mouse failed to escape within 60 s, then it was gently directed to the platform. The mouse was allowed to stay on the platform for 15 s. The time between entering the water and climbing onto the platform was recorded as the latency to escape, and the average latency to escape was calculated for each mouse. The animals were trained for 5 days before the probe trial. The probe trial was performed on the 6th day by removing the platform from the pool. The mice were allowed to swim for 60 s, and the time spent in the target quadrant was recorded.

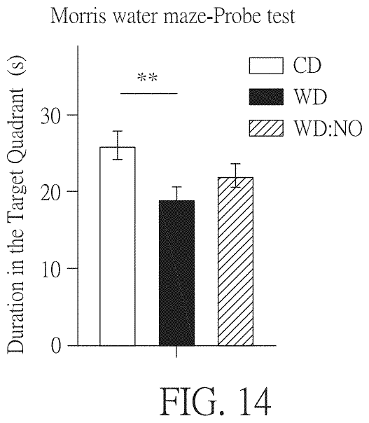

During the acquisition phase of training (days 1-5 during week 17), all groups display a progressive decrease in latency to reach the hidden platform. When the hidden platform is removed during the probe trial, the duration for the aging mice staying in the target quadrant provides a quantitative evaluation of the ability of spatial learning and memory. As shown in FIG. 14, in comparison with the CD group, a significant decrease of duration in the target region displayed by aging mice in WD group supports the cognitive impairment resulted from western diet-induced metabolic syndrome. Of importance, daily treatment of DNIC-1 improves the cognitive impairment in aging mice with metabolic syndrome based on the increase of the duration in the target region featured by WD:NO group (p=0.217 in comparison with WD group).

[Evaluation of Hippocampal Neurogenesis Stimulated by DNIC-1]

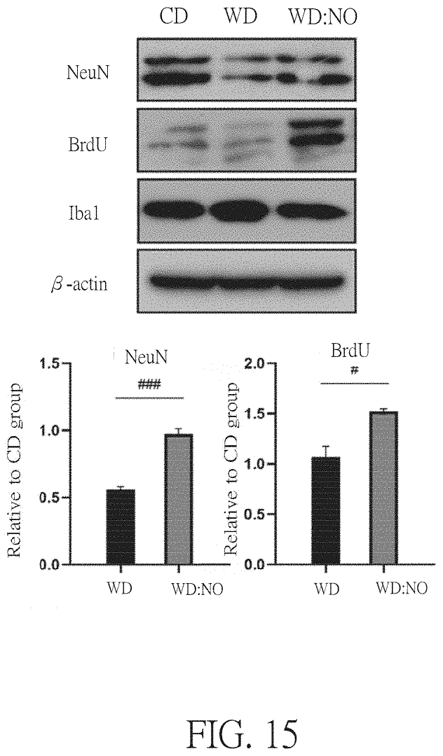

To observe the survival of newborn neurons from hippocampal neurogenesis, aging mice in CD, WD, or WD:NO groups were intraperitoneally injected with BrdU in a daily manner for three consecutive days (0.1 mg of BrdU per gram of body weight). Aging mice were sacrificed after an additional one month before immunoblot analysis on BrdU, NeuN, and Iba1, the analysis results were shown in FIG. 15.

Effects of DNIC-1 on hippocampal neurogenesis were evaluated based on immunoblot analysis on both NeuN and BrdU. In comparison with the aging mice under control diet, western diet-induced obesity and metabolic syndrome imposes a neurotoxic effect and leads to the death of neural cells. Moreover, expression levels of NeuN and BrdU were significantly increased upon daily treatment of DNIC-1 (WD:NO group) when compared with WD group. That is, the pro-neurogenesis effect of nitric oxide released from DNIC-1 triggers the formation of newborn neurons in the hippocampus of aging mice in the WD:NO group. On the other hand, no significant differences between the levels of microglial marker Iba1 in the hippocampus tissues from CD, WD, and WD:NO groups were observed.

In addition, blood samples were collected every four weeks to measure the glutamate oxaloacetate transaminase (GOT), glutamic pyruvic transaminase (GPT), creatinine (CRE), and uric acid (UA). After coagulation, the mouse sera were collected from blood samples by centrifugation at 10,000 g for 20 min at 4° C. and were analyzed.

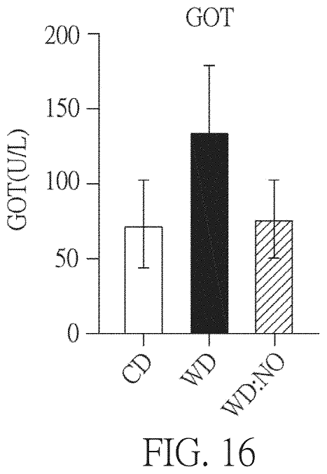

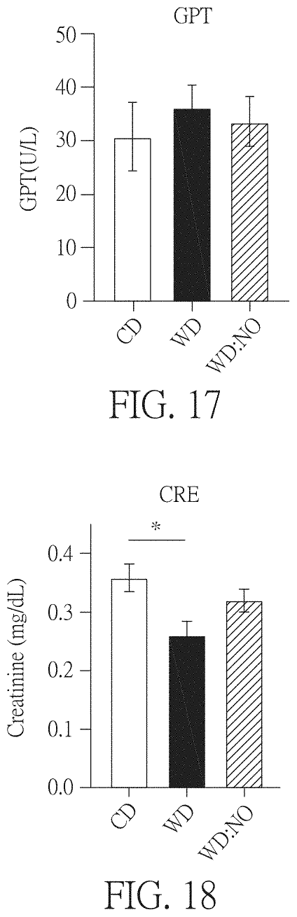

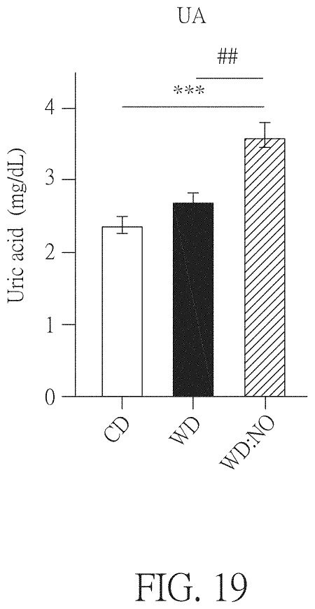

In addition to the therapeutic effect of DNIC-1 on recovery of impaired cognitive ability, as shown in FIG. 16 to FIG. 19, serum chemistry analyses on GOT, GPT, and CRE in CD, WD, and WD:NO groups demonstrate the biocompatibility of DNIC-1 in aging mice under metabolic syndrome, although an elevation of UA in the WD:NO group is observed.

Base on the abovementioned evaluations, the oral pharmaceutical composition of the present invention can be stably absorbed into the circulatory system by the intestine and stomach and release nitric oxide in the brain. Furthermore, the oral administered DNIC-1 can improve neurodegenerative disease and metabolic syndrome, promote hippocampal neurogenesis, and regulate blood sugar.

What is claimed is:

1. An oral pharmaceutical composition, comprising a double nitroso iron complex of formula (I), and a pharmaceutically acceptable excipient: 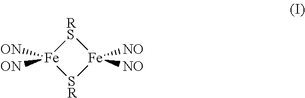
wherein R represents a C₁-C₅ hydroxyl group.

2. The oral pharmaceutical composition of claim 1, wherein the pharmaceutically acceptable excipient is at least one selected from a group consisting of mannitol, xylitol, sorbitol, maltol, maltitol, lactose, sucrose, maltose, and mixture thereof.

3. The oral pharmaceutical composition of claim 2, wherein a dosage form of the oral pharmaceutical composition is a tablet, a capsule, or an oral solution.

4. The oral pharmaceutical composition of claim 1, wherein R represents a C₂ hydroxyl group.

5. A method for delivering nitric oxide to a patient's circulatory system or brain, comprising:
orally administering the oral pharmaceutical composition of claim 1 to the patient.

6. The method of claim 5, wherein delivering nitric oxide to a patient's circulatory system or brain for stimulating neurogenesis or regulating blood sugar.

7. The method of claim 5, wherein delivering nitric oxide to a patient's circulatory system or brain for stimulating neurogenesis refers to a treatment of a neurodegenerative disease or an ischemic stroke.

8. The method of claim 7, wherein the neurodegenerative disease is at least one selected from a group consisting of amyotrophic lateral sclerosis (ALS), Parkinson's disease (PD), Alzheimer's disease (AD), Huntington's disease (HD), frontotemporal dementia (FTD), spinocerebellar ataxias (SCA), Machado-Joseph disease (MJD), Dentatorubropallidoluysian atrophy (DRPLA), spinal and bulbar muscular atrophy (SBMA), and fragile X-associated tremor and ataxia syndrome (FXTAS).

9. The method of claim 6, wherein regulating blood sugar refers to a treatment of diabetes.
